(19) United States
(12) Patent Application Publication
(10) Pub. No.: US 2008/0000801 A1
(43) Pub. Date: Jan. 3, 2008

(54) Automated system, method, and kit for immediate treatment of acute medical condition

(76) Inventor: Robert W. Mackie JR., (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/300,104

(22) Filed: Dec. 13, 2005

Related U.S. Application Data
(60) Provisional application No. 60/635,800, filed on Dec. 13, 2004.

(51) Int. Cl. A61F 17/00; A61B 5/04

(52) U.S. Cl. 206/570; 600/515

(57) ABSTRACT

A system, method, and kit for immediate and initial treatment of an acute medical condition. The system, method, and kit may be self-administered in the event of an acute medical condition.

### CROSS-REFERENCE TO RELATED APPLICATIONS

This application claims priority to U.S. Provisional Application Ser. No. 60/635,800, filed Dec. 13, 2004, which is herein incorporated by reference in its entirety.

### FIELD OF INVENTION

This invention generally relates to methods and treatments for the immediate or initial treatment of acute medical conditions and in particular conditions of acute myocardial infarction and heart attack. More specifically, this invention relates to an automated kit that directs treatments for heart attack and acute myocardial infarction, as well as methods for notification of, and request for assistance from, emergency medical systems and personnel in the setting of heart attack and acute myocardial infarction. The treatments are suitable for self-administration or medical provider supervised administration.

### BACKGROUND OF THE INVENTION

A variety of techniques and drug treatments are well known for the treatment of heart attack and acute myocardial infarction. Immediately reestablishing coronary blood flow with either angioplasty or intravenous thrombolytic treatment is the goal of all current emergency heart attack and myocardial infarction protocols. Reducing the risk of death and limiting myocardial infarction size with adjunctive medical treatment in conjunction with re-establishing coronary blood flow is an important part of treating the heart attack and myocardial infarction patient.

Current adjunctive optimal therapy for emergency treatment of heart attack myocardial infarction and Unstable Coronary Syndromes includes the early administration of aspirin, nitrates for relief of pain and ischemia, and beta-blocker therapy for the reduction of ischemia and prevention of lethal arrhythmias. Early pharmacological or mechanical revascularization is recommended in the American College of Cardiology/American Heart Association (ACC/AHA) guidelines for patients with acute ST elevation infarction. In definite or likely cases of non-ST elevation MI and Unstable Coronary Syndromes in which an initial conservative approach is taken, addition of clopidogrel has been recommended to reduce the incidence of Q wave MI and death. The conjunctive use of the two antiplatelet drugs aspirin (as a cycloxygenase inhibitor) and clopidogrel (as a platelet ADP receptor inhibitor) has been shown to be beneficial in the setting of coronary syndromes treated with percutaneous coronary intervention. The AHA/ACC guidelines list aspirin, nitroglycerin, beta-blockers and clopidogrel as Class I recommendations (known to be beneficial based on prior scientific studies) for immediate oral or intravenous therapy in these settings.

Early treatment of heart attack and myocardial infarction with coronary angioplasty improves coronary blood flow, which in turn reduces myocardial infarction size, improves post infarction cardiac function and reduces post infarction complications. Emergency room pre-thrombolytic treatment with oral clopidogrel in patients suffering heart attack and acute myocardial infarction has been shown to facilitate thrombolytic treatment and improve coronary blood flow.

Generally, the above described prior techniques and drug treatments are not appropriate for self-administration, nor do these prior techniques and drug treatments define a specific treatment sequence to assure proper administration. None of these prior treatments addresses the problem of entering patients into the emergency medical system early in the course of their heart attack or myocardial infarction. Even in the setting of readily available emergency services, including 911 emergency phone numbers, there is often a considerable delay in treatment of Acute Myocardial Infarction. Prior patents exist for wearable devices that will summon emergency medical systems automatically in the event of a cardiac arrest (cessation of a mechanical pulse) when worn by a patient. Prior art devices that can be self-activated in the event of symptoms of a heart attack also exist. For example, one such device employs radiofrequency signals and is described by Christ et al. in U.S. Pat. No. 5,228,449. In addition, U.S. Pat. No. 6,292,687 entitled “Medical Emergency Response and Locator System”, by Lowell et al., describes using a Global Positioning System (GPS) to allow a victim to summon help in the event of a heart attack. However, no current device exists that can summon an emergency response and direct a victim to self administer medications in the event of a heart attack or myocardial infarction.

### SUMMARY OF THE INVENTION

The present invention provides a system and method for treating a victim of acute myocardial infarction and/or its symptoms which includes monitoring the victim's heart rate (pulse) and blood pressure (BP), giving the victim or emergency medical care provider treating the victim an audible and/or visual-monitor screen instruction or instructions to administer essential heart attack medications provided in a self contained kit, and alerting an emergency medical service response team to victim's condition, vital signs, and location through the use of an automated communications system, such as a cellular telephone, for example, and a global positioning satellite system that can locate the automated communications system.

The present invention also includes an emergency treatment kit for treating an acute medical condition, such as myocardial infarction or its symptoms, which comprises at least one medication for treating the acute medical condition, a monitor for monitoring vital signs including a person's blood pressure, heart rate, and/or oxygen saturation, and an automated processing device coupled to the monitor which is capable of monitoring a person's vital sign data received from the monitor and giving an audio and/or visual instruction for administering the medication or medications for treating the acute medical condition and recording the administration of the medication or medications. The automated processing device may also include an automated communications system that is capable of placing an automatic call to “911” and/or a local emergency medical service once the kit is employed for treatment.

The automated processing device may also include a global positioning satellite system that automatically communicates the treated person's location to an emergency response team. This location may be reported during the call to “911” and/or the local emergency medical service. In addition, the automated processing device may also include an automatic timer for recording the date and time of the kit's use as well as the dates and times of medications administered from the kit and the dates and times of blood pressure and heart rate readings from the patient or person being treated. In accordance with one aspect of the invention, the automated processing device may produce a printable treatment record timeline associated with the emergency kit's use.

In accordance with one exemplary embodiment of the emergency treatment kit, the kit is used to treat acute myocardial infarction and/or its symptoms and the kit includes one or more of acetylsalicylic acid, sublingual nitroglycerin, an anticoagulant, a beta-blocker, a calcium blocker, and an antiplatelet medication. Clopidrogel may be used as the anticoagulant and Metoprolol may be used as the beta-blocker.

The present invention is also directed to a system for treating acute mycocardial infarctions and/or its symptoms which includes a processor for receiving and processing data, an automated blood pressure cuff and heart rate monitor coupled to the processor, one or more medications for treating acute myocardial infarction and/or its symptoms, input means for inputting administered medication data to the processor, a memory having an application program coupled to the processor for directing the storage and processing of inputted data, and a display device coupled to the processor and memory for displaying information and instructions derived from the inputted data processed by the processor. The system may further include an automated communications system coupled to the processor for placing an automatic call to “911” and/or an emergency medical service, a global positioning satellite system coupled to the processor for automatically providing the location of the person experiencing the acute myocardial infarction and/or its symptoms, and an automatic timer coupled to the processor for automatically recording dates and times of adapt that is input and processed by the processor.

In one exemplary embodiment of the invention, one or more medications contained in the kit may include one or more of an acetylsalicylic acid, a sublingual nitroglycerin, an anticoagulant, a beta-blocker, a calcium blocker, and an antiplatelet medication.

The system, method, and kit of the present invention for immediate treatment of an acute medical condition such as myocardial infarction is appropriate for both self-administration and supervised administration by a medical provider. The system, method and kit include a defined sequence of use to assure proper administration. The system, method and kit of the present invention also provide for computer programmed adjustment of medication dosages based on a patient's symptom response, pain level, blood pressure and heart rate changes as the patient is treated using the emergency treatment kit.

Additional objects, advantages and other novel features of this invention will be set forth in part in the description that follows and in part will become apparent to those skilled in the art upon examination of the following or may be learned with the practice of the invention. The objects and advantages of this invention may be realized and attained by means of the instrumentalities and combinations particularly pointed out in the appended claims. Still other objects of the present invention will become readily apparent to those skilled in the art from the following description wherein there are shown and described present preferred embodiments of this invention, simply by way of illustration of the present modes best suited to carry out this invention. As it will be realized, this invention is capable of other different embodiments, and its several details, and specific steps, language and package structure are capable of modification in various aspects without departing from the invention. Accordingly, the drawing and descriptions should be regarded as illustrative in nature and not restrictive.

### BRIEF DESCRIPTION OF THE DRAWINGS

The figures illustrate various embodiments of the present invention by way of example, and not by way of limitation. Embodiments of the present invention may include part or all of the features shown in one of these figures, or may include features from two or more figures. Embodiments of the present invention may also include features described in the specification, or elements of features described in the specification. Furthermore, embodiments of the present invention may include features that would be familiar to a person of ordinary skill in the art having studied this document. Thus, a more complete understanding of the present invention may be derived by referring to the detailed description and claims when considered in connection with the drawing figures where like reference numbers refer to similar elements throughout the figures, and

FIG. 1 is a process flow diagram showing an exemplary method of the present invention for immediate treatment of acute myocardial infarction using the kit of the present invention;

FIG. 2 is a process flow diagram showing the order of administration of medications included in an exemplary kit of the present invention for immediate treatment of acute myocardial infarction;

FIG. 3 is a schematic showing an exemplary system of the present invention for immediate treatment of acute myocardial infarction; and

FIG. 4 is a diagram of one exemplary embodiment of the an automated blood pressure cuff and heart rate monitor shown coupled to an automated processing device included in the kit of the present invention for immediate treatment of acute myocardial infarction.

### DETAILED DESCRIPTION

The invention is directed to a system, method, and kit for the self-administration of medications for situations where the patient or user has a critical health emergency, typically a heart attack or suspected heart attack and/or does not have ready access to professional medical assistance. Although this invention can be used for a wide variety of medical conditions or symptoms, the present specific application of this invention is in the treatment of heart attack victims and/or potential heart attack victims. Accordingly, the use or application of this invention as described herein should be treated as examples. The applicant intends that these embodiments be considered as both examples and as descriptions of the best modes of the invention known to the inventor at the time of filing, and not as limiting the scope of patent protection.

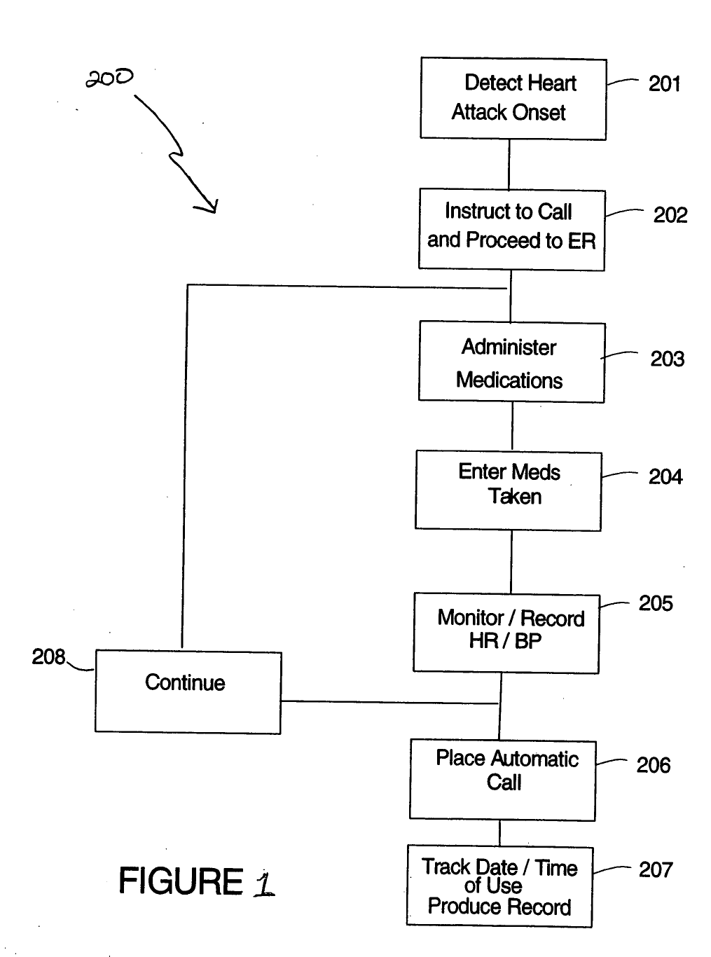

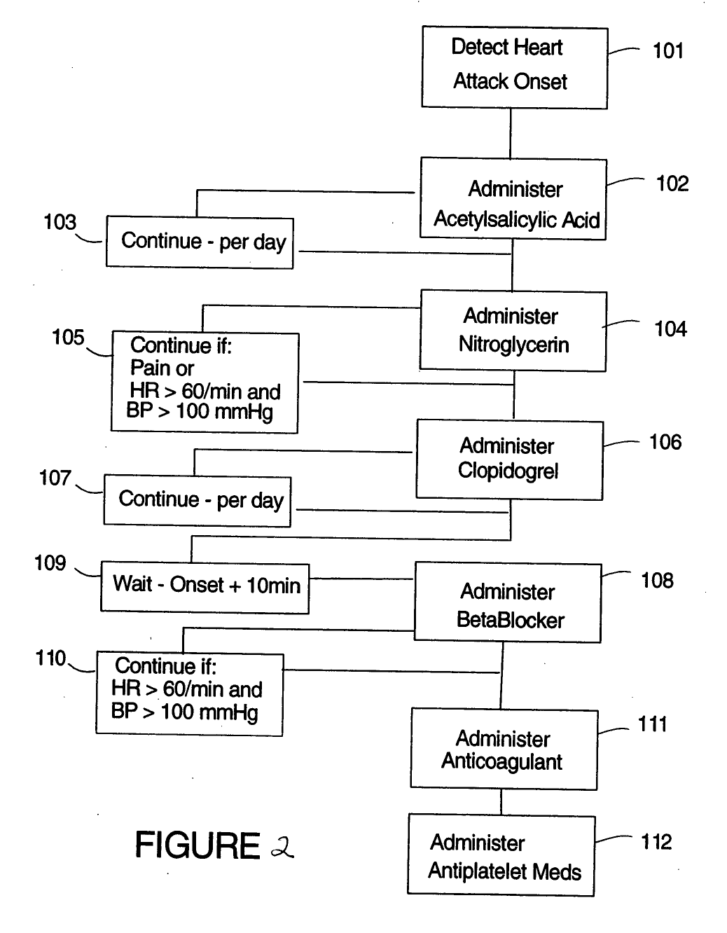

FIG. 1 shows a process flow diagram of the self-administration/lay-bystander treatment process 200. This process makes use of an automatic blood pressure cuff, heart rate monitor and, preferably, a computerized data entry and display device, such as a computer, Personal Data Assistant and the like. Upon detecting the symptoms of a potential heart attack 201, instructions are given 202 by the computerized therapy computer to call for emergency assistance (“911”), the device initiates an emergency computer call to 911 and transmits a GPS location to the 911 center. Instructions are given to proceed to the nearest emergency room regardless of symptom relief. Medications in the kit are administered 203, generally as described above with reference to FIG. 2. Preferably, these medications are administered 203 in the specified order, at the specific time intervals and in response to the patient's measured blood pressure and heart rate. The medications taken are entered 204 into the computerized date entry device, and in the preferred embodiment the blood pressure cuff computerized data entry device will also monitor 205 and keep a record of blood pressure and heart rate measurements during treatment. Treatment continues 208 in response to these blood pressure and heart rate measurements. In one preferred embodiment of this invention, the computerized data entry and display device or the blood pressure cuff may be equipped with Global Positioning Satellite (GPS) and telephone call capabilities, thereby permitting the computerized data entry and display device or blood pressure cuff to place an automatic call to local Emergency Medical Services (EMS). The presently preferred automated blood pressure kit may also include a timer, potentially installed in the data entry and display device, to track the date and time of kit use and to produce a treatment record.

FIG. 2 depicts a process flow diagram showing the order of administration of medications included in an exemplary kit of the present invention for immediate treatment of acute myocardial infarction. When the onset of a heart attack is suspected 101, acetylsalicylic acid (aspirin) is administered 102. The use of aspirin is based on the American Heart Association and American College of Cardiology recommendations for all suspected victims of heart attack, myocardial infarction or unstable angina. Preferably this administration 102 includes four 81 mg tablets, which are chewed and swallowed at the onset of pain. The therapy computer prompts the patient or bystander to respond to an audio and visual prompt to confirm that they have taken the aspirin. Preferably administration of aspirin continues 103 at a dosage of a single 81 mg tablet a day on subsequent days. Aspirin as an anticoagulant has been shown to have the ability to prevent a second heart attack and by taking aspirin during a suspected heart attack, aspirin can significantly reduce damage to the heart and can reduce the formation of potentially dangerous thrombi which can cause further coronary thrombosis or systemic embolism. Aspirin also has anti-inflammatory effects, which may reduce the development of further coronary ulcerations, which may lead to further myocardial infarctions. Following audio and visual written prompts to take aspirin the therapy computer directs the victim to begin taking nitroglycerin 104.

Administration of nitroglycerin 104 is advised by the therapy computer at the onset of the treatment protocol as soon as the victim has answered the prompt that they have taken aspirin. The therapy computer automatically ascertains the victim's heart rate and blood pressure and prompts the victim to take 0.4 mg of sublingual nitroglycerin if the heart rate is above 60 and below 100 beats per minute, and if the systolic blood pressure is above 100 mmHG. If heart rate/blood pressure parameters are outside of this range the computer advises the victim not to take nitroglycerin. If the blood pressure is below 100 mmHg the computer prompts the victim to lie down with legs raised to maintain BP. The computer prompts the patient to avoid nitroglycerin in the event of recent administration of drugs for erectile dysfunction and to avoid nitroglycerin if prior doses have caused syncope or fainting or extreme bradycardia. The therapy computer prompts the patient or bystander to respond to an audio and visual prompt to confirm that they have taken nitroglycerin and automatically reascertains the victim's heart rate and blood pressure two minutes after nitroglycerin is administered.

Immediately following the administration of the first dose of Nitroglycerin, the therapy computer and treatment guidelines advise the administration of a Thienopyridine. In this embodiment of the invention, Clopidogrel is used as an oral anticoagulant, which works in conjunction with aspirin to prevent growth of coronary thrombus and assists in the victim's spontaneous coronary thrombolysis. A Chinese COMMIT trial, which was a randomized placebo-controlled trial of adding Clopidogrel to aspirin in 46,000 acute myocardial infarction patients, proved that the combination of aspirin and clopidogrel reduced mortality and improved outcomes in the setting of myocardial infarction. The Chinese COMMIT trial found a 7% reduction in mortality in acute myocardial infarction when Clopidogrel was added to aspirin. Recently, in the Clopidogrel as Adjunctive Reperfusion Therapy (CLARITY)—thrombolysis in Myocardial Infarction (TIMI) trial, 28 investigators reported their findings from 3491 ST elevation acute myocardial infarction patients receiving intravenous thrombolytic therapy. Patients were randomized to receive a 300 mg loading dose of Clopidogrel or placebo in addition to aspirin, and then 75 mg of Clopidogrel a day. There was a 17% improvement in the degree of TIMI I or II blood flow in patients receiving Clopidogrel. In the PCI-CLARITY Trial, in which patients were treated with 300 mg of Clopidogrel vs. placebo before undergoing coronary angiogram and angioplasty in the setting of acute ST-elevation myocardial infarction, there was a reduction in cardiovascular death, MI or stroke following angioplasty from 6.2% to 3.6%. The loading dose of Clopidogrel in the CLARITY trials was 300 mg. There is known resistance to the antiplatelet effect of Clopidogrel in some patients. In this embodiment of the invention a loading dose of 600 mg Clopidogrel 106 is recommended followed by a daily dose of 75 mg a day 107 of Clopidogrel. The therapy computer advises patients to not take Clopidogrel if they have a history of recent bleeding, trauma, a history of prior intracerebral bleeding or if they are taking other anticoagulant therapy such as coumadin or warfarin. In some instances the invention may be modified to include additional or replacement anticoagulants such as Ximalgatran or Warfarin 111 or may include platelet ADP inhibitors 112 or platelet Gp2b3a inhibitor medications. These other anticoagulants can be helpful in maintaining coronary artery patency and reducing the risk of recurrent myocardial infarction.

The fourth medication in the kit's inventive protocol is a beta-blocker. In this embodiment of the invention, Metoprolol is used and begun 15 minutes after the onset of chest pain 109. Metoprolol was shown in a Metoprolol Acute Myocardial Infarction Trial (MIAMI) to reduce the chances of death and cardiac arrest when begun early in the setting of Acute Myocardial Infarction. The dose of Metoprolol may vary from this embodiment of the invention. The first 15 minutes of treatment with aspirin, nitroglycerin and Clopidogrel, allows for the therapy computer to continue to take the patients heart rate and Blood Pressure every 5 minutes regardless of medications taken, and takes vital signs 2 minutes after each dose of sublingual nitroglycerin. The 15 minute interval allows the computer to take at least four vital sign readings and advises the patient to begin Metoprolol if their Heart Rate is above 60/min and their Blood pressure is above 100 mmHg systolic. If the patient's vital signs fall outside of the desired range of therapy, the computer advises the patient not to begin Metoprolol until their Heart Rate and Blood Pressure fall within the safety range of therapy. The therapy computer advises the patients not to take Metoprolol if they have a history or asthma or a history of allergy to betablockers. In this embodiment of invention Metoprolol is continued every six hours at a dose of 25 mg. The therapy computer automatically takes the patient's BP and heart rate before each dose and then prompts the patient to take or not take Metoprolol based on vital signs 110. In other embodiments of the invention, other betablockers may be used. In patients allergic to betablockers or with a history of asthma, calcium blocker medications may be substituted for betablockers.

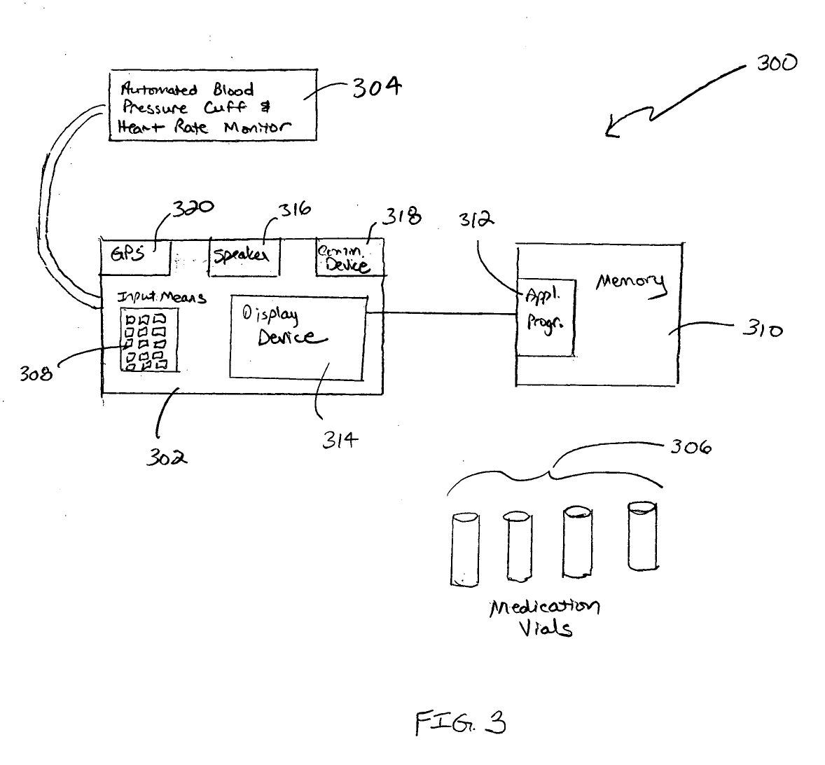

FIG. 3 is a schematic showing an exemplary system 300 of the present invention for immediate treatment of acute myocardial infarction. System 300 includes a processor 302 for receiving and processing inputted data, an automated blood pressure cuff and heart rate monitor 304 coupled to processor 302, and one or more medications 306 for administration to a patient experiencing acute myocardial infarction or its symptoms. System 300 also includes an input means 308, such as a keyboard, keypad or the like, for inputting administered medication data to the processor 302. Input means 308 may or may not be integral with processor 302 but must be coupled in some way to processor 302. System 300 also includes a memory 310 having an application program 312 coupled to processor 302 for directing the storage and processing of inputted data. Data obtained from automated blood pressure cuff and heart rate monitor 304 may be automatically inputted to processor 302. System 300 also includes a display device 314 integral with, and/or coupled to, processor 302 and memory 310 for displaying information and instructions derived from inputted data that is processed by processor 302. System 300 may also include a speaker 316, a communications device 318, such as a cellular phone, and a global positioning system 320.

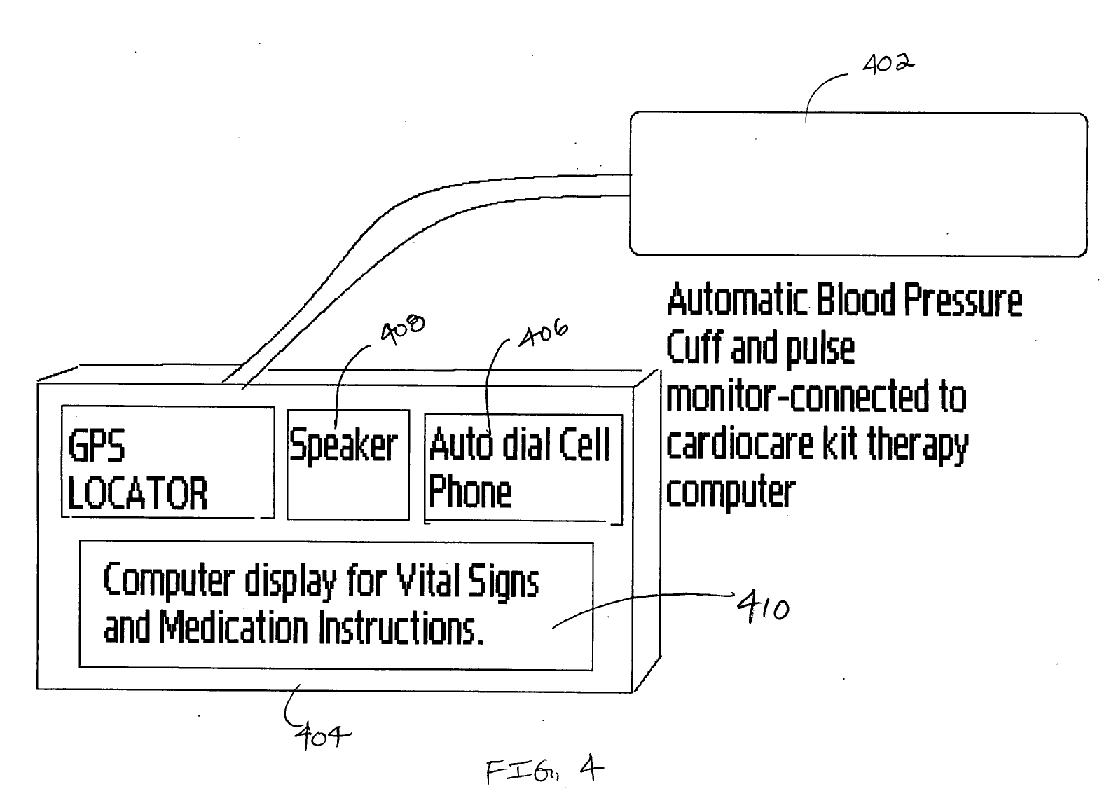

FIG. 4 is a diagram of one exemplary embodiment of the an automated blood pressure cuff and heart rate monitor 402 shown coupled to an automated processing device 404 included in the kit of the present invention for immediate treatment of acute myocardial infarction. Upon powering up the processing device 404, the voice and written instructions guide the patient or bystander through the process of taking blood pressure and heart rate, and automatically dials 911 through a cell phone connection 406. The audio output 408 allows the 911 operators to give advice and to communicate with the victim and/or bystanders. The computer guides the course of therapy included in the kit, and monitors blood pressure and heart rate through the course of therapy. The computer instructs the patient when it is appropriate to continue taking medication and instructs the patient when medications would be contraindicated because of either the presence of a heart rate that is too low or too rapid or a blood pressure that is too low. The computer tracks and stores when medications are taken and automatically takes vital signs routinely throughout the course of therapy. The computer has a UBS port for data download and a visual display 410 of medications taken vital signs throughout the course of therapy.

The described embodiments are to be considered in all respects only as illustrative of the current best modes of the invention known to the inventors at the time of filing this application, and not as restrictive. Although the several embodiments shown here include specific components, drugs (medications), packages and steps, these are provided in order to show examples of the present embodiments of this invention. The specifics of these embodiments are provided to show several examples. This scope of this invention is, therefore, indicated by the appended claims rather than by the foregoing description. All devices and processes that come within the meaning and range of equivalency of the claims are to be embraced as with the scope of this patent.

1. An emergency treatment kit for treating an acute medical condition comprising:
at least one medication for treating an acute medical condition;
a vital sign monitor that determines at least one of a blood pressure, a heart rate, and an oxygen saturation;
an automated processing device coupled to the vital sign monitor wherein the automated processing device is capable of monitoring a patient's vital signs received from the vital sign monitor and giving at least one of an audio and a visual instruction for administering said at least one medication and recording administration of said at least one medication.

2. The emergency treatment kit of claim 1 wherein said automated processing device further comprises an automated communications system capable of placing an automatic call to at least one of “911” and a local emergency medical service.

3. The emergency treatment kit of claim 2 wherein said automated processing device further comprises a global positioning satellite system capable of automatically communicating a patient's location to an emergency response team.

4. The emergency treatment kit of claim 1 wherein said automated processing device further comprises an automatic timer capable of recording a date and time of the kit's use and wherein the automated processing device is capable of producing a treatment record timeline associated with the kit's use.

5. The emergency treatment kit of claim 1 wherein the treatment kit is used to treat acute myocardial infarction or its symptoms and said at least one medication in said kit includes at least one of an acetylsalicylic acid, a sublingual nitroglycerin, an anticoagulant, a beta-blocker, a calcium blocker, and an antiplatelet medication.

6. The emergency medical kit of claim 5 wherein said at least one medication includes an aspirin, a sublingual nitroglycerin, clopidrogel, and metoprolol.

7. A system for treating acute myocardial infarction or its symptoms comprising:
a processor for receiving and processing inputted data;
an automated blood pressure cuff and heart rate monitor coupled to the processor;
at least one medication for administration to a patient experiencing acute myocardial infarction or its symptoms;
input means for inputting administered medication data to the processor;
a memory having an application program coupled to the processor for directing the storage and processing of inputted data; and
a display device coupled to the processor and memory for displaying information and instructions derived from inputted data processed by the processor.

8. The system of claim 7 further comprising an automated communications device coupled to the processor for placing an automatic call to at least one of “911” and a local emergency medical service.

9. The system of claim 8 further comprising a global positioning satellite system coupled to the processor for automatically communicating a location of the patient to an emergency response team.

10. The system of claim 7 further comprising an automatic timer coupled to the processor for automatically recording dates and times of data that is input and processed by the processor.

11. The system of claim 7 wherein said at least one medication includes at least one of an acetylsalicylic acid, a sublingual nitroglycerin, an anticoagulant, a beta-blocker, a calcium blocker, and an antiplatelet medication.

12. A method for providing assistance to a person experiencing an acute myocardial infarction or its symptoms comprising the steps of:
a) providing an automated blood pressure cuff and heart rate monitor coupled to an automated processing device capable of monitoring a person's vital signs automatically received from the blood pressure cuff and heart rate monitor and giving at least one of an audio and a visual instruction for administering at least one medication and recording administration of the medication;
b) positioning the blood pressure cuff and heart rate monitor on a person's body and activating the automated processing device;
c) ascertaining a blood pressure and heart rate for the person from the automated processing device;
d) administering or withholding administration of a medication to the person in response to instructions from the automated processing device;
e) inputting to the automated processing device an amount of the medication administered to the person; and
f) repeating steps c through e until instructed otherwise by the automated processing device.

13. The method of claim 12 wherein the step of providing an automated blood pressure cuff and heart rate monitor coupled to an automated processing device includes the step of providing an automated processing device with a communications system capable of placing an automatic call to at least one of “911” and an emergency medical service and the method further comprises the step of automatically placing that call.

14. The method of claim 13 wherein the step of providing an automated processing device with a communications system includes the step of providing an automated processing device with a global satellite system capable of determining a location of the person and the step of placing an automatic call to at least one of “911” and an emergency medical service includes the step of providing the person's location.

15. The method of claim 12 further comprising the step of providing a treatment record including dates and times of all administered medications and dates and times of all blood pressure and heart rate readings taken from the person.

16. The method of claim 12 wherein the step d comprises the step of administering or withholding administration of acetylsalicylic acid and step e comprises the step of inputting to the automated processing device an amount of acetylsalicylic acid administered to the person.

17. The method of claim 16 wherein step f comprises the steps of:
ascertaining a blood pressure and a heart rate of the person from the automated processing device;
administering or withholding administration of a sublingual nitroglycerin to the person in response to instructions from the automated processing device; and
inputting to the automated processing device an amount of the sublingual nitroglycerin administered to the person.

18. The method of claim 17 wherein step f further comprises the steps of:
ascertaining a blood pressure and a heart rate of the person from the automated processing device;
administering or withholding administration of an anticoagulant to the person in response to instructions from the automated processing device; and
inputting to the automated processing device an amount of the anticoagulant administered to the person.

19. The method of claim 18 wherein step f further comprises the steps of:
g) ascertaining a blood pressure and a heart rate of the person from the automated processing device;
h) administering or withholding administration of a beta-blocker to the person in response to instructions from the automated processing device; and
i) inputting to the automated processing device an amount of the beta-blocker administered to the person.

20. The method of claim 19 wherein wherein the steps of g through i are repeated at predetermined intervals until instructed otherwise by the automated processing device.

21. The method of claim 20 further comprising the steps of:
ascertaining a blood pressure and a heart rate of the person from the automated processing device;
administering or withholding administration of at least one of an anticoagulant and an antiplatelet to the person in response to instructions from the automated processing device; and
inputting to the automated processing device an amount of at least one of the anticoagulant and the antiplatelet administered to the person.
